(19) United States
(12) Patent Application Publication
(10) Pub. No.: US 2023/0263420 A1
(43) Pub. Date: Aug. 24, 2023

(54) ELECTRONICS ARRANGEMENT FOR A WEARABLE ARTICLE

(71) Applicant: Prevayl Innovations Limited, Manchester (GB)

(72) Inventor: Reiss Cashmore, Manchester (GB)

(21) Appl. No.: 17/769,559

(22) PCT Filed: Nov. 13, 2020
(86) PCT No.: PCT/GB2020/052900

§ 371 Date: Apr. 15, 2022

(30) Foreign Application Priority Data
Nov. 15, 2019 (GB) 1916653.7

(51) Int. Cl. A61B 5/0531; A61B 5/00; A61B 5/024; A61B 5/308; A61B 5/313; G06N 20/00

(52) U.S. Cl. CPC A61B 5/0531; A61B 5/7264; A61B 5/02416; A61B 5/308; A61B 5/313; A61B 5/6802; G06N 20/00

(57) ABSTRACT

The electronics arrangement comprising a processor (201); and a memory (203), the at least one memory (203) storing instructions, the instructions, when executed by the processor (201), cause the processor (201) to perform operations comprising: obtaining a current version of a machine-learned model; obtaining first data from at least one sensor (211) of the wearable article (20); and employing the current version of the machine-learned model to generate an inference using the first data. The processor (201) may determine whether to update the machine-learned model based on the generated inference. The processor (201) may comprise a hardware accelerator. The processor (201) may cause data to be transmitted to a base station for updating the machine-learned model.

### CROSS-REFERENCE TO RELATED APPLICATIONS

This application claims priority from United Kingdom Patent Application number 1916653.7 filed on 15 Nov. 2019, the whole contents of which are incorporated herein by reference.

### BACKGROUND

The present invention is directed towards a method and electronics arrangement for a wearable article, and in particular towards performing local machine learning operations on the wearable article or a base station associated with the wearable article.

Wearable articles comprise sensors to measure properties such as the physiological, psychological, biochemical, environmental and behavioural traits of a user wearing the wearable device. The data sensed by the wearable articles are used in machine-learning applications and in particular to train models for recognizing properties of the user and/or the activities the user is undertaking. To train machine-learned models from data sensed by a number of wearable articles, it is conventionally required that the wearable articles all transmit data to a central server. The central server has the computational resources to train machine-learned models using large data sets received from many wearable articles.

One problem with this approach is that it requires potentially sensitive data to be transmitted to and held at a central server. This can present potential privacy and security issues especially if the central server is compromised or run by an untrustworthy entity. Moreover, many people may be uncomfortable with sharing their sensitive data with the central server and thus may be reluctant to use such wearable articles. This reduces the amount of data available for training machine-learned models and thus may reduce the accuracy of any resultant models trained on the central server. This is particularly problematic when models are trained for generating inferences that will have a positive effect on individuals by providing inferences related to their health, lifestyle, fitness and performance.

It is an object of the present disclosure, to provide machine-learning functionality for wearable articles that allows for machine-learned models to be trained from data sensed by a number of different wearable articles without requiring the data to be transmitted to a central server.

### SUMMARY

According to the present disclosure there is provided a method and apparatus as set forth in the appended claims. Other features of the invention will be apparent from the dependent claims, and the description which follows.

A first aspect of the present disclosure provides a method performed by an electronics arrangement for a wearable article. The method comprises the following steps:
(a) obtaining a current version of a machine-learned model;
(b) obtaining first data from at least one sensor of the wearable article;
(c) employing the current version of the machine-learned model to generate an inference using the first data;
(d) determining whether to update the current version of the machine-learned model based on the generated inference.

Advantageously, the electronics arrangement performs on-device machine-learning and determines whether to update the current version of the machine-learned model based on the inference result. Performing these operations on-device avoids the need to transmit potentially sensitive data to a remote server for model inference and updating.

Step (a) may comprise receiving the current version of the machine-learned model from an external computer apparatus such as a server.

Step (d) may comprise comparing a confidence level of the generated inference to a first predetermined threshold.

If the confidence level of the generated inference is greater than or equal to the first predetermined threshold, the method may further comprise (e) transmitting the first data and the generated inference for the first data to a base station for the wearable article. Advantageously, the first data and the generated inference is transmitted to a base station that is able to determine an update to the machine-learned model. The base station is able to perform the relatively computationally intense task of performing the model update operation which may, in some circumstances, be undesirable to perform on the wearable article due to considerations such as size, power, and computational resources. The base station is still local to the wearable article which means that sensitive data is kept locally and is not transmitted to a remote central server.

If the confidence level of the generated inference is greater than or equal to the first predetermined threshold, the method may further comprise (e) updating the current version of the machine-learned model using the first data obtained from the wearable article. Advantageously, the wearable article performs on-device model updating which means that sensitive data is kept locally and is not transmitted to a remote central server.

Updating the current version of the machine-learned model may comprise updating the machine-learned model using a backwards propagation of errors approach, a weight imprinting approach, and/or a transfer learning approach. Other model update techniques may be used in accordance with the present disclosure.

The method may further comprise (f) transmitting updated machine-learned model data to an external computer apparatus. Advantageously, the updated machine-learned model data is transmitted to an external computer apparatus such as a server. The server may aggregate the updated machine-learned model data to generate an updated model. This enables a model to be updated by a server using data sensed by wearable articles without requiring that the server has direct access to the data sensed by the wearable articles.

Step (b) may comprise obtaining first data and second data from the at least one sensor of the wearable article.

The first data may comprise activity data sensed by the at least one sensor of the wearable article. The generated inference may comprise an activity classification. The activity classification may relate to whether the user is walking, standing, sitting, running, cycling or performing any other form of activity that the machine-learned model has been trained to recognise. The first data may comprise physiological data sensed by the at least one sensor of the wearable article. The generated inference may comprise a physiological and/or behavioural classification. The physiological classification may relate to classifying the users cardiac state, respiratory state, stress levels, emotional state, fatigue, and for classifying whether the user is drowsy. The first data may comprise biometric identification data sensed by the at least one sensor of the wearable article. The generated inference may comprise a biometric identification classification.

The inference may be generated based on data obtained from a plurality of different sensors of the wearable article and may also consider data from other sources external to the wearable article.

The at least one sensor may comprise at least one of an optical sensor, force sensor, electrical sensor temperature sensor and, acoustic sensor. The optical sensor may comprise a photoplethysmography, PPG, sensor. The force sensor may comprise at least one of an accelerometer, a magnetometer and a gyroscope. The electrical sensor may comprise at least one of an electropotential potential sensor and an electroimpedance sensor, optionally wherein the electropotential sensor comprises electrocardiography, ECG, sensor and/or a electromyography, EMG, sensor, optionally wherein the electroimpedance sensor comprises a skin conductance sensor.

A second aspect of the present disclosure provides an electronics arrangement for a wearable article. The electronics arrangement comprises at least one processor and at least one memory storing instructions. The instructions, when executed by the processor, cause the processor to perform the method of the first aspect of the disclosure.

The electronics arrangement may further comprise a communicator for communicating with an external computer apparatus. The electronics arrangement may further comprise a power source arranged to power the electronics arrangement. The electronics arrangement may further comprise at least one sensor, optionally wherein the at least one sensor comprises at least one of an optical sensor, force sensor, electrical sensor and temperature sensor, acoustic sensor.

The electronics arrangement may comprise a removable electronic module for the wearable article, the electronics module comprises the at least one processor and the at least one memory, the electronics module is configured to be releasably mechanically coupled to the wearable article.

The at least on processor may comprise a hardware accelerator arranged to employ at least a component of the machine-learned model. The hardware accelerator may comprise one or a combination of a graphics processing unit, a field-programmable gate array, a dedicated application specific integrated circuit, a visual processing unit, a tensor processing unit, a neural processing unit, and a neural processing engine. The at least one processor may comprise an application processor. The hardware accelerator and the application processor may be employed on the same semiconductor package.

A third aspect of the present disclosure provides a wearable article comprising the electronics arrangement of the second aspect of the present disclosure.

The wearable article may be any form of electronic device which may be worn by a user such as a smart watch, necklace, bracelet, or glasses. The wearable article may be a textile article. The wearable article may be a garment. The garment may refer to an item of clothing or apparel. The garment may be a top. The top may be a shirt, t-shirt, blouse, sweater, jacket/coat, or vest. The garment may be a dress, brassiere, shorts, pants, arm or leg sleeve, vest, jacket/coat, glove, armband, underwear, headband, hat/cap, collar, wristband, stocking, sock, or shoe, athletic clothing, swimwear, wetsuit or drysuit. The wearable article/garment may be constructed from a woven or a non-woven material. The wearable article/garment may be constructed from natural fibres, synthetic fibres, or a natural fibre blended with one or more other materials which can be natural or synthetic. The yarn may be cotton. The cotton may be blended with polyester and/or viscose and/or polyamide according to the particular application. Silk may also be used as the natural fibre. Cellulose, wool, hemp and jute are also natural fibres that may be used in the garment. Polyester, polycotton, nylon and viscose are synthetic fibres that may be used in the garment. The garment may be a tight-fitting garment. Beneficially, a tight-fitting garment helps ensure that any sensors of the garment are held in contact with or in the proximity of a skin surface of the wearer. The garment may be a compression garment. The garment may be an athletic garment such as an elastomeric athletic garment.

A fourth aspect of the present disclosure provides a system. The system comprises an electronics arrangement comprising at least one processor and at least one memory storing instructions, the instructions, when executed by the processor, cause the processor to perform operations, the operations comprising:
(a) obtaining a current version of a machine-learned model;
(b) obtaining first data from at least one sensor of the wearable article;
(c) employing the current version of the machine-learned model to generate an inference using the first data;
(d) determining whether to update the current version of the machine-learned model based on the generated inference such as by comparing a confidence level of the generated inference to a first predetermined threshold; and
(e) if the confidence level of the generated inference is greater than or equal to the first predetermined threshold, transmitting the first data and the generated inference for the first data to a base station for the wearable article.

The system further comprises a base station for a wearable article. The base station comprises at least one processor and at least one memory storing instructions. The instructions, when executed by the processor, cause the processor to perform operations, the operations comprising:
(f) obtaining a current version of a machine-learned model;
(g) receiving the first data and the generated inference from the electronics arrangement for the wearable article; and
(h) updating the current version of the machine-learned model using the first data obtained from the wearable article and the generated inference.

A fifth aspect of the present disclosure provides an electronics arrangement for a wearable article. The electronics arrangement comprising: a plurality of processors, the plurality of processors comprising an application processor, and a hardware accelerator; at least one memory storing instructions, the instructions, when executed by the plurality of processors, cause the plurality of processors to perform operations comprising:
(a) obtaining a current version of a machine-learned model;
(b) obtaining first data from at least one sensor of the wearable article; and
(c) employing the current version of the machine-learned model to generate an inference using the first data.

Advantageously, the present disclosure provides an electronics arrangement for a wearable article that is adapted to perform computationally efficient machine-learning using a hardware accelerator. This enables on-device machine-learning to performed despite size, power and other constraints of the wearable article. Beneficially, this avoids the need to transmit potentially sensitive data to a central server.

The electronics arrangement may comprise a removable electronics module configured to be releasably mechanically coupled to the wearable article. The electronics module may comprise the plurality of processors and the at least one memory.

The at least one sensor may be provided in the wearable article separately to the electronics module.

A sixth aspect of the present disclosure provides a wearable article comprising the electronics arrangement of the fifth aspect of the present disclosure.

A seventh aspect of the present disclosure provides a base station for a wearable article. The base station comprises a plurality of processors. The plurality of processors comprise an application processor, and a hardware accelerator; at least one memory storing instructions, the instructions, when executed by the plurality of processors, cause the plurality of processors to perform operations comprising:
(a) obtaining a current version of a machine-learned model;
(b) obtaining first data from at least one sensor of the wearable article; and
(c) employing the current version of the machine-learned model to generate an inference using the first data.

An eighth aspect of the present disclosure provides computer-implemented method comprising the following steps:
(a) obtaining a current version of a machine-learned model;
(b) obtaining first data from a wearable article;
(c) determining whether to update the current version of the machine-learned model using the first data obtained from the wearable article; and
(d) in response to determining to update the current version of the machine-learned model, updating the current version of the machine-learned model using the first data obtained from the wearable article.

A ninth aspect of the present disclosure provides a device comprising: a processor; and a memory storing instructions that, when executed by the processor, cause the processor to perform operations, the operations comprising:
(a) obtaining a current version of a machine-learned model;
(b) obtaining first data from a wearable article;
(c) determining whether to update the current version of the machine-learned model using the first data obtained from the wearable article; and
(d) in response to determining to update the current version of the machine-learned model, updating the current version of the machine-learned model using the first data obtained from the wearable article.

A tenth aspect of the present disclosure provides a system comprising: a server; and a device according to the ninth aspect of the present disclosure. The server is arranged to transmit a machine-learned model to the device.

An eleventh aspect of the present disclosure provides a method performed by an electronics arrangement for a wearable article, the method comprising the following steps:
(a) obtaining a current version of a machine-learned model;
(b) obtaining first data from a sensor of the wearable article;
(c) determining whether to update the current version of the machine-learned model using the first data; and
(d) in response to determining to update the current version of the machine-learned model, transmitting the first data to a base station.

A twelfth aspect of the present disclosure provides an electronics arrangement for a wearable article. The electronics arrangement comprising: a processor; and a memory storing instructions that, when executed by the processor, cause the processor to perform operations, the operations comprising:
(a) obtaining a current version of a machine-learned model;
(b) obtaining first data from a sensor of the wearable article;
(c) determining whether to update the current version of the machine-learned model using the first data; and
(d) in response to determining to update the current version of the machine-learned model, transmitting the first data to a base station.

A thirteenth aspect of the present disclosure provides a base station for a wearable article, the base station comprising: a processor; and a memory storing instructions that, when executed by the processor, cause the processor to perform operations. The operations comprising:
(a) obtaining a current version of a machine-learned model;
(b) obtained first data from the wearable article; and
(c) updating the current version of the machine-learned model using the first data obtained from the wearable article.

A fourteenth aspect of the present disclosure provides a system comprising: an electronics arrangement of the twelfth aspect of the present disclosure; and a base station of the thirteenth aspect of the present disclosure.

A fifteenth aspect of the present disclosure provides a device. The device comprises: a power source; a communicator; at least one processor; and at least one memory storing instructions that, when executed by the processor, cause the processor to perform operations, the operations comprising:
(a) obtaining heart-rate data from a heart-rate sensor of the wearable article and motion data from a motion sensor of the wearable article; and
(b) employing a machine-learned model to generate an inference using the heart-rate data and motion data.

A sixteenth aspect of the present disclosure provides a wearable article comprising the device of the fifteenth aspect of the disclosure.

A seventeenth aspect of the present disclosure provides a method performed by a device as of the fifteenth aspect of the present disclosure. The method comprising: (a) obtaining heart-rate data and motion data from the wearable article; and (b) employing a machine-learned model to generate an inference using the heart-rate data and motion data.

An eighteenth aspect of the present disclosure provides a computer-implemented method comprising:
(a) obtaining a machine-learned model for generating an inference using data sensed by a wearable article;
(b) compressing the machine-learned model;
(c) transmitting the compressed version of the machine-learned model to a plurality of devices;
(d) receiving updated machine-learned model data from one or more of the devices, the updated machine-learned model data being derived from data sensed by at least one sensor of a wearable article; and
(e) using the updated machine-learned model data to generate an updated version of the machine-learned model.

A nineteenth aspect of the present disclosure provides a server comprising: at least one processor; at least one memory storing instructions, the instructions, when executed by the at least one processor, cause the at least one processor to perform the method of the eighteenth aspect of the present disclosure.

A twentieth aspect of the present disclosure provides a system comprising: a server comprising at least one processor; and at least one memory storing instructions, the instructions, when executed by the at least one processor, cause the at least one processor to perform operations, the operations comprising:
(a) obtaining a machine-learned model for generating an inference using data sensed by a wearable article;
(b) compressing the machine-learned model;
(c) transmitting the compressed version of the machine-learned model to a plurality of devices;
(d) receiving updated machine-learned model data from one or more of the devices, the updated machine-learned model data being derived from data sensed by at least one sensor of a wearable article; and
(e) using the updated machine-learned model data to generate an updated version of the machine-learned model; and
a plurality of devices, each comprising at least one processor; and at least one memory storing instructions, the instructions, when executed by the at least one processor, cause the at least one processor to perform operations, the operations comprising:
(f) receiving the compressed version of the machine-learned model;
(g) obtaining first data from a wearable article; and
(h) updating the compressed version of the machine-learned model using the first data obtained from the wearable article.

The present disclosure is not limited to wearable articles and instead may be applied to other forms of devices such as non-wearable textile articles. Such textile articles may include upholstery, such as upholstery that may be positioned on pieces of furniture, vehicle seating, a wall or ceiling decor, among other examples.

### BRIEF DESCRIPTION OF THE DRAWINGS

Examples of the present disclosure will now be described with reference to the accompanying drawings, in which:

FIG. 1 is simplified schematic diagram of an example system according to aspects of the present disclosure;

FIG. 2 is a simplified schematic diagram of an example wearable article according to aspects of the present disclosure;

FIG. 3 is a simplified schematic diagram of a plurality of example wearable articles communicating with a base station according to aspects of the present disclosure;

FIG. 4 is a simplified schematic diagram of an example system according to aspects of the present disclosure;

FIG. 5 is an example swim-lane flow diagram for an example method according to aspects of the present disclosure;

FIG. 6 is a simplified schematic diagram for another example system according to aspects of the present disclosure;

FIG. 7 is a flow diagram for an example method according to aspects of the present disclosure;

FIG. 8 is a flow diagram for another example method according to aspects of the present disclosure;

FIG. 9 is a flow diagram for yet another example method according to aspects of the present disclosure; and

FIG. 10 is a flow diagram for yet another example method according to aspects of the present disclosure.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise.

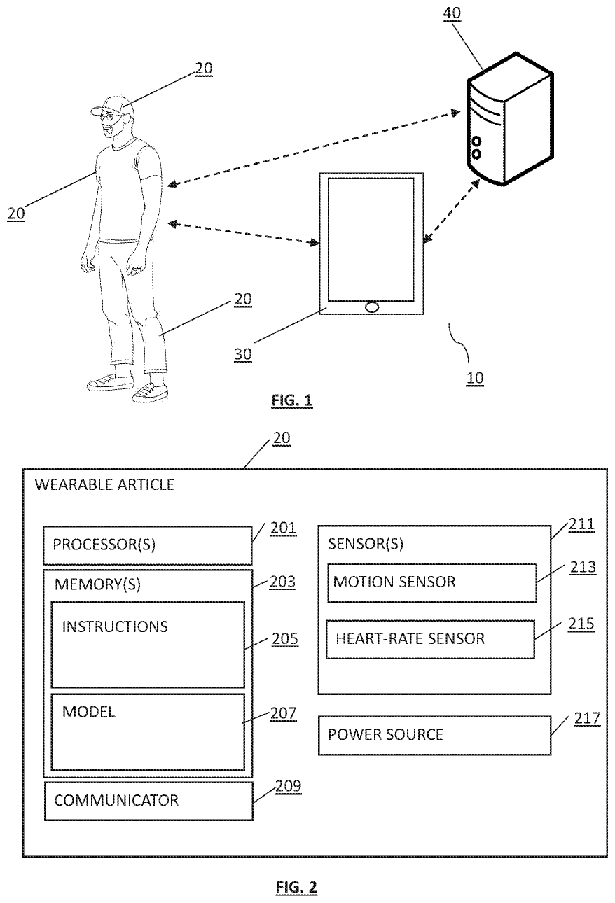

Referring to FIG. 1, there is shown an example system 10 according to aspects of the present disclosure. The system 10 comprises a plurality of wearable articles 20 worn by a user. The plurality of wearable articles 20 are garments and in particular, a top, bottoms, and headwear. The system 10 comprises a server 40. The wearable articles 20 communicate with the server 40 over a wireless network such as a cellular network. The system 10 comprises a base station 30 for the wearable articles 20. The wearable articles 20 communicate with the base station 30 over a wired or wireless communication protocol. The wearable articles 20 communicate with the server 40 over a wired or wireless communication protocol. The wearable articles 20 are not required to communicate with the server 40 in all examples of the present disclosure and may instead communicate only with the base station 30 or indirectly with the server 40 via the base station 30. The wearable articles 20 comprises sensors that measure signals and transmits the same to the server 40 and/or the base station 30. Generally, the sensors comprise biosensors which are arranged to measure biosignals of the user.

It is a general objective of the present disclosure to train machine-learned models using data sensed by the wearable articles 20. Conventionally, this requires the wearable articles 20 to transmit the data to the server 40. The server 40 then uses the data received from the plurality of wearable articles 20 as training data to train or update a machine-learned model or a plurality of machine-learned models. A problem with these existing approaches is that potentially sensitive data measured by the wearable articles 20 is transmitted, stored, and analysed on a remote server 40. This presents potential privacy and security issues. Moreover, depending on the number of wearable articles 20 transmitting data, the amount of data, and the type of data, there may be excessive demands on bandwidth and computational resources of the server 40.

To overcome problems associated with existing approaches, the present disclosure locally updates the machine-learned models on the wearable articles 20 or the base stations 30 communicatively coupled to the wearable articles 20. By locally updating the machine-learned models, potentially sensitive data measured by the wearable articles 20 is not required to be transmitted to, stored or analysed on the remote server 40. Instead, only updated machine-learned model data may be transmitted to the server 40. The updated machine-learned model data does not convey sensitive information or at least any sensitive information that may be computationally easily extracted from the updated model data. The server 40 updates the machine-learned model using the updated machine-learned model data. The server 40 is able to receive updated machine-learned model data received from a number of wearable articles/base stations to generate an updated machine-learned model that reflects the updates generated by the plurality of wearable articles 20/base stations 30. The present disclosure therefore enables a federated learning approach that has been particularly adapted for wearable articles considering, amongst other factors the hardware, battery and size constraints of existing wearable articles.

Referring to FIG. 2, there is shown a simplified schematic diagram of an example wearable article 20 according to aspects of the present disclosure. The schematic diagram shows the electronics components of the wearable article. These electronics components may be referred to as an electronics arrangement. The electronics arrangement for the wearable article 20 comprises one or more processors 201, at least one memory 203, a communicator 209, one or more sensors 211, and a power source 217. The memory 203 stores instructions 205 and a machine-learned model 207 amongst other data. The instructions 205, when executed by the processor 201, cause the processor 201 to perform operations. The communicator 209 enables communication with the base station 30 and and/or the server 40 (FIG. 1) over one or more networks. The one or more sensors 211 are arranged to measure signals from the user wearing the wearable article 20 or external to the user wearing the wearable article 20. In the example of FIG. 2, the sensors 211 comprise a motion sensor 213 which may be an inertial measurement unit and a heart-rate sensor 215 which may be an electrocardiography, ECG, sensor. It will be appreciated that the present disclosure is not limited to this particular combination of sensors 213 and instead additional or other forms of sensors may be provided. Motion sensors 213 and heart-rate sensors 215 and, in particular, the combination therefore are advantageous as the resultant motion and heart-rate data can be used to derive a number of useful physiological inferences. That is, a limited number of sensors 213, 215 can be used to generate a relatively large number of inferences.

The motion sensor 213 may comprise a force sensor. A force sensor refers to a sensor that measure the force that affects the sensor. The force may be due to movement in the case of an accelerometer such as a 3-axis accelerometer, the Coriolis force in the case of a gyroscope, the Earth's magnetic field in the case of a magnetometer, or air pressure in the case of a barometer. The force sensor may comprise an accelerometer such as a 3-axis accelerometer. An accelerometer can measure forces produced by muscular induced movement of the wearer. The force sensor may comprise a magnetometer which measures the strength of the magnetic field and thus can be used to derive the strength and direction of the Earth's magnetic field. The magnetometer may measure the strength of the magnetic field along three axes. The sensor 104 may comprise a gyroscope. Gyroscopes are able to measure the attitude and rotation of different body parts of the user depending on their positioning in the wearable article 20 and the location of the wearable article 20 on the body.

The sensors 211 may comprise an optical sensor. An optical sensor may measure the amount of ultraviolet, visible, and/or infrared light in the environment. The optical sensor may comprise a photoplethysmographic (PPG) sensor. PPG sensors measure blood volume changes within the microvascular bed of the wearer's tissue. PPG sensors use a light source to illuminate the tissue. Photodetectors within the PPG sensor measure the variations in the intensity of absorbed or reflected light when blood perfusion varies.

The sensor 211 may comprise an electrical sensor. An electrical sensor may measure the electrical activity of a part of the body or how a current changes which it is applied to the body. An electrical sensor may perform biopotential measurements. An example biopotential sensor is an electrocardiogram, ECG, sensor that measures the electrical activity of the heart. An electrical sensor may perform bioimpedance measurements. That is, the electrical sensor may comprise a bioimpedance sensor. Bioimpedance measurements may be obtained by performing different impedance measurements between different points on user's body at different frequencies. An example bioimpedance sensor is a galvanic skin response sensor that measures the skin conductance. The skin conductance varies depending on the amount of moisture (induced by sweat) in the skin. Sweating is controlled by the sympathetic part of the nervous system, so it cannot be directly controlled by the subject. The skin conductance can be used to determine body response against physical activity, stress or pain.

The sensor 211 may comprise a temperature sensor such as a skin temperature sensor. A skin temperature sensor may comprise a thermopile arranged to capture infrared energy and transform it into an electrical signal that represents the temperature. The sensor 211 may comprise a humidity sensor such as to measure skin surface wetness. The sensor 211 may comprise an acoustic sensor. The acoustic sensor may comprise a microphone. The acoustic sensor may be arranged to measure the user's voice. The acoustic sensor may be arranged to measure other (typically low power) sounds emitted from the user, such as the user's heart. The wearable article 20 may comprise other sensors for measuring other signals these sensors may be biosensors for measuring biosignals of the wearer. “Biosignals” may refer to any signal obtained from a living being that can be measured and monitored.

In some examples, the processor 201, memory 203, and communicator 209 may be provided as an electronics module for the wearable article 20. The electronics module may be arranged to form a detachable mechanical and/or electronic connection with the wearable article 20. The electronics module may be disposable in a pocket of the wearable article such as a garment pocket. This may enable the electronics module to be removed from the rest of the wearable article 20 and connected to the base station 30 (FIG. 1) for charging and/or data transfer. The power source 217 may also be provided in the electronics module. The sensors 211 may be provided in the wearable article 20 separately to the electronics module or may be incorporated into the electronics module. Some of the sensors 211 such as the motion sensor 213 may be provided in the electronics module while others are provided separately in the wearable article 20. The electronics module is not required to be removable from the remainder of the wearable article 20 in all aspects of the present disclosure. The electronics components may be integrated into the wearable article 20.

The electronics module is preferably removable from the wearable article 20 and is configured to be releasably mechanically coupled to the wearable article 20. The mechanical coupling of the electronics module to the wearable article 20 may be provided by a mechanical interface such as a clip, a plug and socket arrangement, etc. The mechanical coupling or mechanical interface may be configured to maintain the electronics module in a particular orientation with respect to the wearable article 20 when the electronics module is coupled to the wearable article 20. This may be beneficial in ensuring that the electronics module is securely held in place with respect to the wearable article 20 and/or that any electronic coupling of the electronics module and the wearable article 20 (or a component of the wearable article 20) can be optimized. The mechanical coupling may be maintained using friction or using a positively engaging mechanism, for example.

Beneficially, the removable electronics module may contain all of the components required for data transmission and processing such that the wearable article 20 only comprises the sensor components and communication pathways. In this way, manufacture of the wearable article 20 may be simplified. In addition, it may be easier to clean a wearable article 20 which has fewer electronic components attached thereto or incorporated therein. Furthermore, the removable electronics module may be easier to maintain and/or troubleshoot than embedded electronics. The electronics module may be configured to be electrically coupled to the wearable article 20.

It may be desirable to avoid direct contact of the electronics module with the wearer's skin while the wearable article 20 is being worn. It may be desirable to avoid the electronics module coming into contact with sweat or moisture on the wearer's skin. The electronics module may be provided with a waterproof coating or waterproof casing. For example, the electronics module may be provided with a silicone casing. It may further be desirable to provide a pouch or pocket in the garment to contain the electronics module in order to prevent chafing or rubbing and thereby improve comfort for the wearer. The pouch or pocket may be provided with a waterproof lining in order to prevent the electronics module from coming into contact with moisture.

The wearable article 20 performs on-device machine-learning using the machine-learned model 207 stored in the memory 203. In order to employ the machine-learned model 207, the wearable article 20 may implement a machine learning platform. The machine-learning platform may be stored locally in the memory 203 of the wearable article 20. When executed by the processor 201, the machine-learning platform enables the wearable article 20 to perform machine-learning functions. The machine-learning functions may be performed using one or more machine learning engines implemented locally on the wearable article 20. Applications running on the wearable article 20 can communicate with the machine-learning platform via one or more application programming interfaces (APIs). An inference API may be provided to enable the machine-learning platform to obtain inferences using the machine-learned model 207 and data sensed by the sensor 211. The machine-learning platform may also obtain instructions for running the model to obtain inferences and model parameters. The machine-learning platform may obtain the inference according to the instructions and model parameters by interacting with the machine learning engine to cause implementation of the model by the engine. The inference may be a physiological inference relating to one or more physiological properties of the user wearing the wearable article 20 as derived from the data. The inference may relate to the likelihood of a user wearing the wearable article 20 having a pre-set property. An updating API may be provided to enable the machine-learning platform to update the machine-learned model 207 based on training data. The machine-learning platform may also obtain instructions for updating the model and model parameters. The wearable article 20 is not required to perform both model inference and updating, and instead may only perform model updating, model inference, or may perform neither model inference nor updating.

Throughout the present disclosure, updating the machine-learned model may refer to various training or learning techniques. The updating may refer to re-training a machine-learned model using the training data (e.g. from scratch), but this is not required in all implementations and is generally less preferred due to the amount of time, and computational resources required. This is a problem for wearable articles 20 which due to size and power constraints typically have limited available computational resources. Updating the machine-learned model may comprise updating the machine-learned model using a backwards propagation of errors approach. Updating the machine-learned model may comprise updating the machine-learned model using a weight imprinting approach. Updating the machine-learned model may comprise updating the machine-learned model using a transfer learning approach. Transfer learning involves retraining an existing model. This can involve retraining the whole model by adjusting the weights across the whole network, but accurate results can also be obtained by removing the final layer of the machine-learned model that performs classification and training a new layer on top. The training of the last layer may be performed by using weight imprinting on the last layer or backpropagation on the last layer. A number of generalization techniques such as weight decays and dropouts may be employed to improve the generalization capability of the models being updated. The updating of the machine-learned model generates updated machine-learned model data. The updated machine-learned model data may comprise an updated version of the machine-learned model. The updated machine-learned model data may comprise an update vector. The update vector may be in the form of a gradient which represents a local update to the machine-learned model. Updating the machine-learned model may comprise performing an ensemble learning operation using a plurality of machine-learned models stored locally on the device (e.g. the wearable article or base station). These machine-learned models may be locally updated based on the generated inferences, and the updated machine-learned models may be aggregated such as by using ensemble learning techniques to generate a updated machine-learned models. Aspects of the present disclosure may use an ensemble learning technique known as stacking, super learning or stacked regression. In stacking, a meta-learner, also known as a blender or final predictor, is trained to find the optimal combination of base learners.

After the processor 201 generates the inference, the processor 201 may then determine whether to update the machine-learned model 207. In some examples, the processor 201 uses a user input to determine whether to update the machine-learned model 207. The user input may be an input from the user confirming whether or not they have the pre-set property associated with the generated inference. If the user input confirms that the user has the pre-set property, then the data may be labelled as training data associated with the pre-set property and used to update the model. If the user input confirms that the user does not have the pre-set property, then the data may be not be used for updating the model. The user input may be a touch input, voice input, gesture, or other form of user input received via the wearable article 20 or a device associated with the wearable article 20. In some examples, the processor 201 uses the generated inference to determine whether to update the machine-learned model. For example, the processor 201 may determine whether the confidence level of the generated inference is greater than or equal to a first predetermined threshold. If the confidence level is greater than or equal to the first predetermined threshold, then the processor 201 determines to update the machine-learned model 207 using the data as training data. In this way, data which is determined with a high confidence level to be associated with a pre-set property may be used to update the machine-learned model 207. The first predetermined threshold may represent a confidence level of 90% or higher, 80% or higher, 70% or higher, or 60% or higher for example. Of course, any other threshold value may be set as appropriate by the skilled person in the art. In some examples, if the confidence level is less than the first predetermined threshold but greater than or equal to a second predetermined threshold, then the processor 201 may use a user input to determine whether to update the machine-learned model 207.

In some examples, if the processor 201 determines to update the machine-learned model 207, then the processor 201 may update the machine-learned model 207. In these implementations, it will be appreciated that the base station 30 (FIG. 1) and the server 40 (FIG. 1) may not be required. The server 40 may still be provided to enable the wearable article 20 to receive an initial version of the machine-learned model and communicate updated machine-learned model data to the server 40. This may enable the wearable article 20 to participate in federated learning with other wearable articles. The base station 30 in preferred implementations may still be present to provide additional machine-learning and/or charging capabilities.

In some examples, if the processor 201 determines to update the machine-learned model 207 then the wearable article 20 transmits the data and the inference to the base station 30 (FIG. 1) so as to be used as training data to update a machine-learned model stored on the base station 30. The machine-learned model may be the same as the model 207 stored on the wearable device 20 or may be a different (e.g. updated) machine-learned model. This approach is beneficial as the computationally intensive task of model updating is not performed on the wearable article 20 but rather a base station 30. Due to size, battery and portability constraints, wearable articles 20 typically have limited computational capability to perform machine-learning operations. Transmitting the training data to the base station 30 therefore allows for local and secure model updating to be performed for the wearable device 20 even if the wearable device 20 does not have the computational or power requirements to perform on-device model re-training. Performing inference operations on the wearable device 20 may be beneficial in allowing for inferences to be generated without requiring the wearable article 20 to be in communication with the base station 30.

The processor 201 of the wearable article 20 comprises an application processor and an AI hardware accelerator. The AI hardware accelerator may perform at least a component the machine-learning inference and updating operations. The AI hardware accelerator may comprise a graphics processing unit (GPU), a field-programmable gate array (FPGA), a dedicated AI accelerator application specific integrated circuit (ASIC), a visual processing unit (VPU), a tensor processing unit (TPU), a neural processing unit (NPU), a neural processing engine, a co-processor; a controller; or combinations of the processing devices described above. processing devices can be embedded within other hardware components such as, for example, a sensor. Beneficially, the Al hardware accelerator reduces the time required for the wearable article 20 to perform machine-learning inference and updating operations compared to conventional application processors.

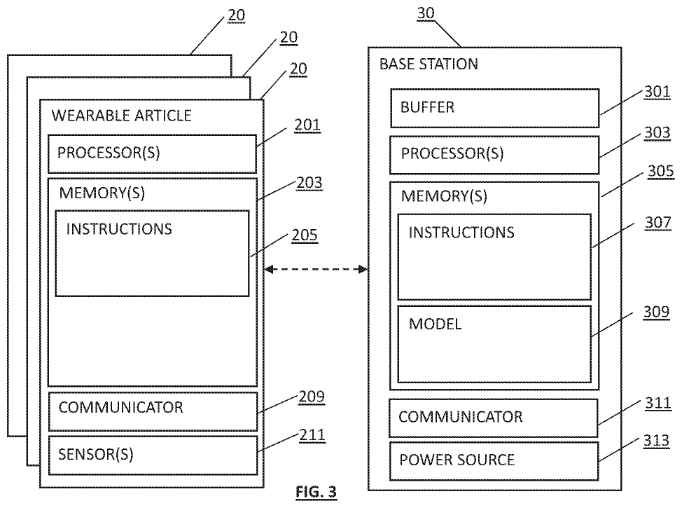

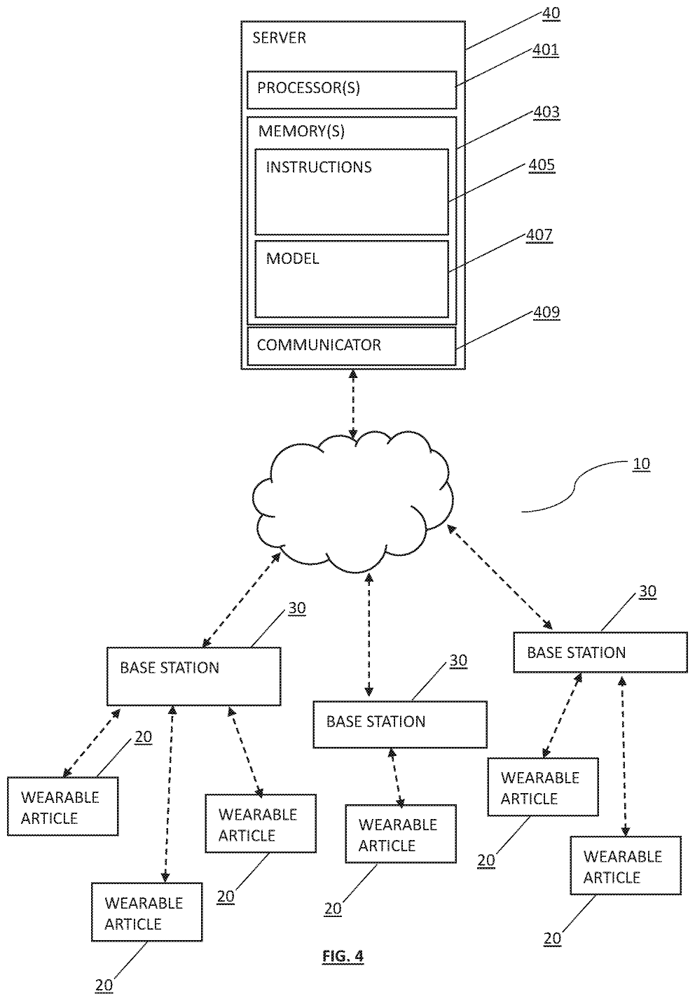

Referring to FIG. 3, there is shown a simplified schematic diagram of a local environment where a plurality of wearable articles 20 are coupled to a base station 30 for the wearable articles 20. The wearable articles 20 may be the same as the wearable articles 20 shown in the example of FIG. 2 and like reference numerals have been used to indicate like components. The wearable articles 20 shown in FIG. 3, however, do not store machine-learned models in memory 203. This is because these wearable articles 20 do not perform local inference. The wearable articles 20 of FIG. 3 are therefore not required to perform on-device machine learning and may not have any machine-learning capabilities or models stored in the memory 203.

The base station 30 acts a docking station for the wearable articles 20 or just the electronics modules of the wearable articles 20. The wearable articles 20 may be coupled to the base station 30 to transfer data and receive power for charging a power source of the base station 30. The wearable articles 20 may establish communication sessions with the base station 30 and then may transfer and, in particular, stream data to the base station 30. The communication session may be established by physically connecting the wearable articles 20 to the base station 30 over a wired connection which may, for example, use the Universal Serial Bus (USB) protocol. Alternatively, the communication session may be established by the wearable articles 20 establishing a wireless communication session with the base station 30. The wireless communication session may be over a near field, short range or local wireless communication protocol such as Bluetooth, or WiFi. Of course any other form of wired or wireless communication may be used as appropriate by the skilled person in the art to enable the wearable article 20 to transfer data to the base station 30.

The base station 30 is not limited to docking/charging stations for wearable articles and may be another form of electronic device such as a user electronic device/mobile phone. Any electronic device capable of communicating with a server and/or a wearable device over a wired or wireless communication network may function as a base station in accordance with the present invention. The base station may be a wireless device or a wired device. The wireless/wired device may be a mobile phone, tablet computer, gaming system, MP3 player, point-of-sale device, or wearable device such as a smart watch. A wireless device is intended to encompass any compatible mobile technology computing device that connects to a wireless communication network, such as mobile phones, mobile equipment, mobile stations, user equipment, cellular phones, smartphones, handsets or the like, wireless dongles or other mobile computing devices. The wireless communication network is intended to encompass any type of wireless such as mobile/cellular networks used to provide mobile phone services. The base station 30 may be a base station for a cellular network. This enables the present disclosure to take advantage of edge computing on the cellular network. Beneficially, a base station for a cellular network is still local to the user and avoids the transmission and storage of data on a remote server.

The base station 30 comprises a buffer 301, one or more processors 303, at least one memory 305, communicator 311 and power source 313. The memory 305 can store instructions 307 and a model 309 amongst other data. The instructions 307, when executed by the processor 303, cause the processor 303 to perform operations. The buffer 301 is arranged to temporarily store data received from the wearable articles 20. The communicator 311 enables communication with the wearable articles 20 and the server 40 over one or more networks. The power source 313 is arranged to transfer power from the base station 30 to the wearable articles 20. The base station 30 is not required, in all implementations, to be fixed or electrically connected to a mains power source. The base station 30 may be a portable device.

The wearable articles 20 transmit data to the base station 30 so that the base station 30 may perform inference and model updating operations using the machine-learned model 309 stored in the memory 305. In order to employ the machine-learned model 309, the base station 30 may implement a machine learning platform. The machine-learning platform may be stored locally in the memory 305 of the base station 30. When executed by the processor 303, the machine-learning platform enables the base station 30 to perform machine-learning functions for the base station 30. The machine-learning functions may be performed using one or more machine learning engines implemented locally on the base station 30. Applications running on the base station 30 can communicate with the machine-learning platform via one or more application programming interfaces (APIs). An inference API may be provided to enable the machine-learning platform to obtain data from the buffer 303 and obtain inferences based on the obtained data from the machine-learned model 309. The machine-learning platform may also obtain instructions for running the model to obtain inferences and model parameters. The machine-learning platform may obtain the inference according to the instructions and model parameters by interacting with the machine learning engine to cause implementation of the model by the engine. An updating API may be provided to enable the machine-learning platform to update the machine-learned model 309 based on training data. The machine-learning platform may also obtain instructions for updating the model and model parameters.

The base station 30 receives the streamed data and temporarily stores the data in a buffer 301. The buffer 301 may be volatile memory. Volatile memory means that the data is not permanently retained by the base station 30 and is lost if the base station 30 powers off. The processor 303 reads data from the buffer 301 and employs the machine-learned model 309 stored in the memory 305 to generate an inference using the data read from the buffer 301. The data may be removed from the buffer 301 once it is read. After the processor 303 generates the inference, the processor 303 then determines whether to update the machine-learned model 309. In some examples, the processor 303 uses a user input to determine whether to re-train the machine-learned model 309. In some examples, the processor 303 uses the generated inference to determine whether to update the machine-learned model. These approaches are performed in substantially the same way as described above for the wearable article 20 of FIG. 2.

The processor 303 may then store the updated model 309 in the memory 305. The memory 305 may be non-volatile memory 305. In this way, for subsequent data read from the buffer 301, the re-trained machine-learned model 309 is employed to generate an inference. Each time data is read from the buffer 301, an inference may be generated, and the processor 303 may determine whether to re-train the machine-learned model 309. In this way, the machine-learned model 309 may be frequently or continuously re-trained as data is read from the buffer 301.

The processor 303 may sequentially read data from the buffer 301, generate an inference, determine whether to update the machine-learned model 309 and, if required, update the machine-learned model 309 using the data as training data. In some examples, however, the base station 30 may pool training data together before updating the machine-learned model 309. For example, the processor 303 may read data from the buffer 301, employ the machine-learned model 309 stored in the memory 305 to generate an inference using the data read from the buffer 301, and determine from the generated inference whether to use the data as training data to update the machine-learned model 309. If the processor 303 determines to use the data as training data, the data may be added to the pool of training data. The processor 303 may then proceed to read the next data from the buffer 301. Once a condition has been reached, the base station 30 then updates the machine-learned model 309 using the pool of training data. The condition may be any of a sufficient amount of training data has been pooled, all the data has been read from the buffer 301, the base station 301 is in an idle state, no wearable devices 20 are connected to the base station 30, the base station 30 is plugged into a power source, a predetermined threshold of power is available, a scheduled time has been reached or any other condition as may be appropriately selected by the skilled person.

The processor 301 of the base station 30 comprises an application processor and an Al hardware accelerator. The Al hardware accelerator may perform the machine-learning inference and updating operations. The Al hardware accelerator may comprise a graphics processing unit (GPU), a field-programmable gate array (FPGA), a dedicated Al accelerator application specific integrated circuit (ASIC), a visual processing unit (VPU), a tensor processing unit (TPU), a neural processing unit (NPU), a neural processing engine, a co-processor; a controller; or combinations of the processing devices described above. Processing devices can be embedded within other hardware components such as, for example, a sensor.

FIG. 3 shows that a plurality of wearable articles 20 are connected to the base station 30 and are transmitting data to the base station 30. The wearable articles 20 may sequentially transmit data to the base station 30 or simultaneously transmit data to the base station 30. The base station 30 may comprise a plurality of buffers 301 for temporarily storing data from the plurality of wearable articles 30. The base station 30 may comprise a single buffer 301 for temporarily storing data from the plurality of wearable articles 30. The buffer 301 may use a non-locking structure which means that data from a plurality of different sources is able to be written to the buffer 301 at the same time. In addition, while data is being written to the buffer 301, data may also be simultaneously read from the buffer 301 such as to be used in the employed machine-learned model. The use of non-locking buffer 301 is beneficial as it allows for faster retraining times and reduces the amount of time required for the wearable articles 20 to be communicating with the base station 30. In some examples, while the wearable articles 20 are connected to the base station 30 and transmitting data to the base station 30, the processor 303 may not read data from the buffer 301. Instead, the processor 301 may wait until the wearable articles 20 have finished transmitting data to the base station 30 or another condition is met such as the buffer 301 reaching a full state.

In some examples, the base station 30 determines or obtains updated machine-learned model data for a plurality of wearable articles and aggregates the updated machine-learned model data to generate an updated machine-learned model.

Referring to the examples of FIGS. 2 and 3, the updated machine learned model 207, 309 as determined by either the wearable article 20 or the base station 30 may be used for subsequent local inferences using data sensed by the sensor 211 of the wearable article 20. In some examples, the communicator 209 of the wearable article 20 or the communicator 311 of the base station 30 may transmit updated machine-leaned model data to the server 40.

Referring to FIG. 4, there is shown a schematic representation of an example system 10 according to aspects of the present disclosure. The system 10 comprises a server 40, a plurality of base stations 30 communicatively connected to the server 40 over one or more networks, and a plurality of wearable articles 20 connected to different ones of the base stations 30. Each of the base stations 30 may be provided at a geographically distinct location such as different homes of different users. The wearable articles 20 connected to each base station 30 may represent the wearable articles 20 associated with a user or a group of users within each of the geographic locations.

The server 40 comprises one or more processors 401, at least one memory 403 and a communicator 409. The memory 403 stores instructions 405 and a machine-learned model 407 amongst other data. The instructions 405, when executed by the processor 401, cause the processor 401 to perform operations. The communicator 409 enables communication with the base stations 30 over one or more networks such as the internet. The base stations 30 and the wearable articles 20 are the same as those described in relation to FIGS. 2 and 3.

The processor 401 of the server 40 obtains the machine-learned model 407 and controls the communicator 409 to transmit the machine-learned model 407 to the base stations 30. Prior to the transmission of the machine-learned model 407, the processor 501 may implement a model compressor to compress the machine-learned model 407. Compressing the machine-learned model 407 reduces the size of the machine-learned model 407. Beneficially, this reduces the amount of data that has to be transmitted to the base stations 30 over the network. Moreover, this reduces the amount of data that has to be stored on the base stations 30. The base stations 30 may only have a limited amount of storage compared to the server 40. The model compressor may perform quantization of one or more weights of the machine-learned model where the quantization error introduced by the quantization can be compensated by later quantization errors. The model may be compressed using a tree-pruning approach. The model is not required to be compressed in all aspects of the present disclosure. The model may be converted into a format suitable for the local device such as the base station 30 or wearable article 20. For example, the model may be converted into Plain Old Java Object (POJO) for deployment on a Java application running on the local device.

The base stations 30 receive the compressed machine-learned model 407 from the server 40 and store the same in their respective memory. As and when the wearable articles 20 are brought into communication with their respective base stations 30, the wearable articles 20 stream data to the base stations 30. The base stations 30 update the machine-learned models using local data received from the local wearable articles 20. The base stations 30 transmits updated machine-learned model data to the server 40. The server 40 then aggregates the updated machine-learned model data received from the base stations 30 to generate an updated machine-learned model.

Aggregating the updated machine-learned model data may comprise using ensemble learning techniques to ensemble updated multiple machine-learned model data together into one machine-learned model, which preferably represents the optimal combination of all of the machine-learned models. Example ensemble learning techniques include max voting, averaging, weighted averaging, stacking, blending, bagging and boosting. Bagging algorithms include bagging meta-estimator and random forest. Boosting algorithms include AdaBoost and Gradient Boosting, LightGBM and XGBM. Of course, other methods of aggregating updated machine-learned model data are within the scope of the present disclosure.

The present disclosure therefore enables for the federated learning of a machine-learned model from data sensed by a plurality of wearable articles 20 potentially spread over a wide geographic location. The present disclosure beneficially does not transmit potentially sensitive data from the wearable articles 20 to the server 40 and instead performs the model-retraining locally at a local base station 30. The local base station 30 may only temporarily retain data received from the wearable articles 20 and may not store the data received from the wearable articles 20 in a permanent or persistent form. The wearable articles 20 may be required to connect to or dock with the base station 30 for a number of reasons such as data offload and charging. The present disclosure advantageously provides additional functionality for the base station by incorporating machine-learning functions into the base station.

The server 40 may initially define and generate the machine-learned model based on a schema received from a third party. For example, the third party may request that the server 40 train a model based on a defined schema. One example schema may specify that a model be trained for identifying the “risk of injury” of “people under 30”. The schema may of course specify any other or additional parameters for the model. The server 40 may then train an initial machine-learned model using training data or otherwise obtain an initial machine-learned model. The initial machine-learned model may have been trained using only a limited pool of users (e.g. 10s to 100s of users). It is desirable to generate a more refined and accurate machine-learned model using a larger pool of users (e.g. 1000s plus). The present disclosure achieves this by distributing the model to the plurality of base stations 30. The model may only be distributed to base stations 30 that satisfy the criteria of the schema, e.g. “people under 30”. Otherwise, the base station 30 may determine whether the user wearing the wearable article 20 satisfies the criteria before performing the model updating operation. The base station 30 may determine this from data received from the wearable article 20.

The server 40 may not be a single computing device, and instead may be distributed over a plurality of computing devices. That is, the server 40 may be a distributed computing system such as a cloud server 40.

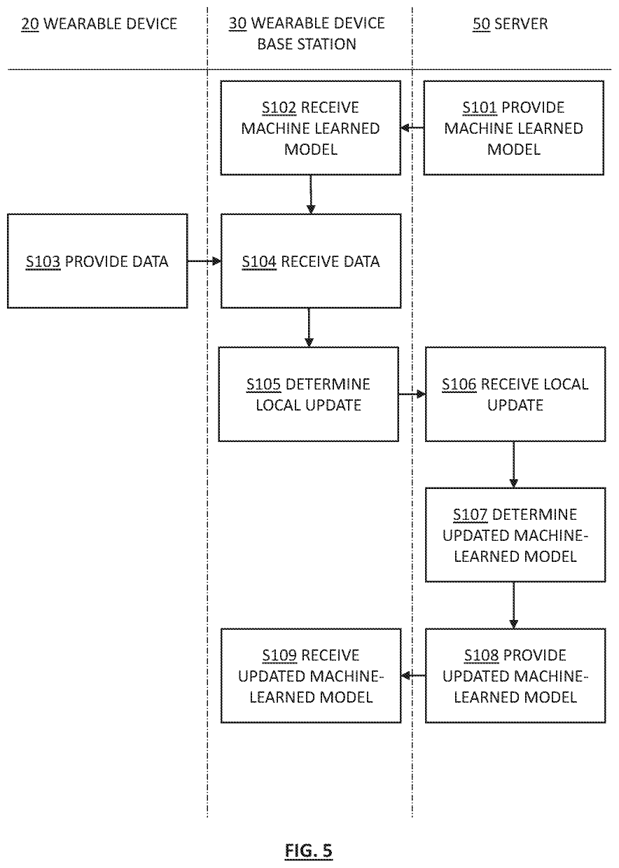

Referring to FIG. 5, there is shown a swim lane flow diagram of an example operation performed by the system of FIG. 3. Step S101 of the method comprises the server 50 providing the machine-learned model to the base station 30 for the wearable articles 20 The base station 30 receives the machine-learned model in step S102 and stores the machine-learned model in a memory. In step S103 of the method, the wearable device 20 provides data to the base station 30. The base station 30 receives the data in step S104 and determines a local update to the machine-learned model in step S105. The server 50 receives the local update to the machine-learned model in step S106 and determines an updated machine-learned model in step S107. In step S108, the server 50 provides the updated machine-learned model to the base station 30 which receives the updated machine-learned model in step S109.

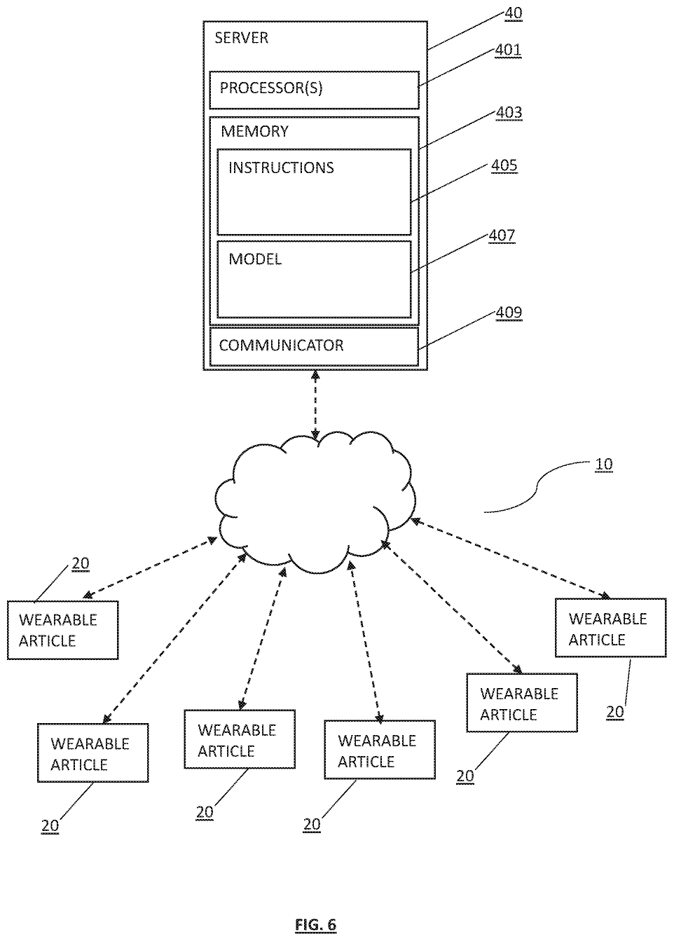

Referring to FIG. 6, there is shown another example system 10 according to aspects of the present disclosure. The system 10 comprises a server 40, and plurality of wearable articles 20 communicatively connected to the server 40 over one or more networks. In this example, the wearable articles 20 receive the model from the server 40 and perform local updating of the model prior to transmission of the update data for the model to the server 40. The server 40 then aggregates the received updated model data to generate an updated model. In this example, the base stations 30 are therefore not required.

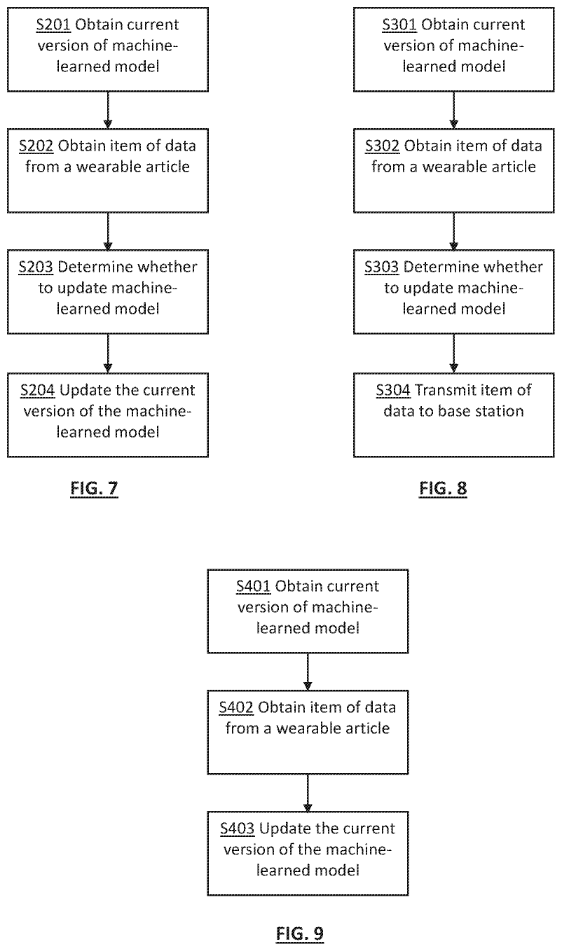

Referring to FIG. 7, there is shown a flow diagram for an example computer-implemented method according to aspects of the present disclosure. Step S201 comprises obtaining a current version of a machine-learned model. Step S202 comprises obtaining data from a wearable article. Step S203 comprises determining whether to update the current version of the machine-learned model using the data obtained from the wearable article. Step 204 comprises, in response to determining to update the current version of the machine-learned model, updating the current version of the machine-learned model using the data obtained from the wearable article.

Referring to FIG. 8, there is shown a flow diagram for an example computer-implemented method according to aspects of the present disclosure. Step S301 comprises obtaining a current version of a machine-learned model. Step S302 comprises obtaining data from a sensor of the wearable article. Step S303 comprises determining whether to update the current version of the machine-learned model using the data. Step 304 comprises in response to determining to update the current version of the machine-learned model, transmitting the data to a base station.

Referring to FIG. 9, there is shown a flow diagram for an example computer-implemented method according to aspects of the present disclosure. Step S401 of the method comprises obtaining a current version of a machine-learned model. Step S402 of the method comprises obtaining data from the wearable article. Step S403 of the method comprises updating the current version of the machine-learned model using the data obtained from the wearable article.

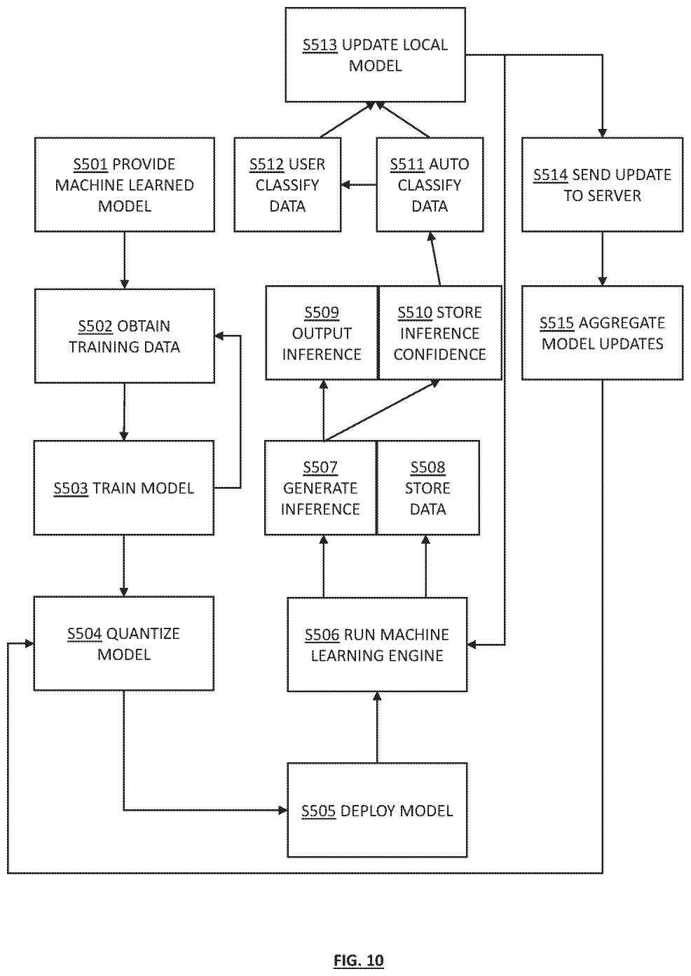

Referring to FIG. 10, there is shown a flow diagram for an example computer-implemented method according to aspects of the present disclosure.

Step S501 of the method comprises providing a machine-learned model. The machine-learned model is built to perform an inference to recognise a pre-set property. In this example, the machine-learned model is built to recognise a motion state of a user wearing a wearable article based on motion data sensed by one or more motion sensors of the wearable article.

Step S502 of the method comprises obtaining training data for training the machine-learned model. The training data may comprise data obtained and labelled from a number of different users. These users may have opted into sharing their data with the server. For example, a plurality of users may wear wearable articles comprising motion sensors and perform a number of different actions such as sitting, running, standing, cycling and jumping. This data may be transmitted to the server. At the server side, the data may be labelled to identify the action that the data relates to. The data may first be clustered using an unsupervised learning procedure such as hierarchical clustering, k-Means clustering, or gaussian mixture models. The data clusters may then be labelled. The user may label the data prior to transmission to the server such that the server is not required to perform labelling operations. The training data may additionally or separately comprise simulated data. This may mean that computer simulations of different user actions are generated and used to provide training data.

Step S503 of the method comprises training the machine-learned model using the training data obtained in step S502. Steps S502 and S503 may be repeated as additional training data is obtained so as to refine the machine-learned model.

Step S504 of the method comprises quantizing the model. In this step, the server employs a model compressor to perform a quantization of the model and therefore reduce the size of the model prior to transmission to the local device. The quantizing of the model may mean that high-precision parameters of the model such as the model weights and activation outputs are converted into lower-precision parameters. As an example, original 32-bit floating-point number parameters are converted to 8-bit fixed-point numbers. This reduces the size of the model and enables the model to be run faster on a local device. Although the model parameters of the quantized model are less precise, the inference accuracy of the model may not be significantly affected. The server may also format the quantized model such that it has a format suitable to be run on the local device. This may involve compiling the model to make it compatible with the hardware of the local device. In some situations, a model may comprise operations that are supported by an AI hardware accelerator of the local device and may also comprise operations that are unsupported by the AI hardware accelerator. The supported operations may be compiled to run on the AI hardware accelerator of the local device while the unsupported operations may be compiled to run on the application processor of the local device. Generally, it is preferably to perform operations using the AI hardware accelerator as performing operations on the application processor can slow the inference speed.

The quantized and compiled model is transmitted to the local device. The local device in this example is a wearable article but may also be a base station for a wearable article in some examples of the present disclosure. In step S505 of the method the machine-learned model is deployed on the wearable article, and in step S506 of the method a machine-learning engine is run local on the device to perform machine-learning functions.

Step S507 of the method comprises generating an inference using the machine-learning engine and the deployed machine-learned model. The inference is generated using data sensed by the wearable article and in this particular example from motion data sensed by one or more motion sensors of the wearable article. The generated inference indicates the confidence level that the motion data indicates that a user has performed a certain action. In step S508, the data input to the machine-learned model is stored locally on the wearable article.

Step S509 comprises outputting the inference. The inference may be output by the wearable article. The wearable article may comprise an output unit for outputting the inference. The output unit may comprise an audio output unit (e.g. a speaker), a display or a haptic feedback unit. Other forms of output unit are within the scope of the present disclosure. The inference may be output on a separate electronic device in communication with the wearable article such as a mobile phone. Step S510 comprises storing the confidence level of the inference. The confidence level of the inference is stored as metadata associated with the relevant data stored in step S508. The inference may also be stored.

In step S511, the wearable article determines whether the determined confidence level for the inference is sufficiently high to use the data as training data to update the local model on the wearable article. This involves the wearable article comparing the determined confidence level to a first predetermined threshold. If the confidence level is determined to be greater than or equal to the first predetermined threshold, then the wearable article determines to use the data as training data to update the local model. If the confidence level is less than the first predetermined threshold, then in step S512 the user may be prompted to classify the data themselves. This may involve the user providing a user input to confirm the activity they are performing. For example, the user may issue the vocal statement “I am running” to confirm that the motion data sensed by the wearable article indicates that they are running. The vocal statement may be detected by an audio input unit of the wearable article. The input may be provided via a separate electronic device in communication with the wearable article such as a mobile phone. Step S512 may only be performed if the confidence level is less than the first predetermined threshold and greater than or equal to the second predetermined threshold. Data associated with a confidence level less than the second predetermined threshold may not be used to update the local model.

The consequence of steps S511 and S512 is that labelled data is obtained for updating the local machine-learned model in step S513. The updating may be performed on the wearable article or may be performed on a base station for the wearable article. Performing the local updating on a base station is preferred in some implementations due to size and power considerations. The method then returns to step S506 so that inferences for subsequent data are performed using the updated local model.

Once the local inferring and updating operations are completed, updated model data is transmitted to the server in step S514. In step S515 the server aggregates the received updated model data from a plurality of wearable articles/base stations to generate an updated global model. The method then returns to step S504 and the updated global model is quantized prior to distribution to wearable articles for local updating.

In examples of the present disclosure, the transmitting of the data from the wearable device to the base station may only be performed until a criteria has been reached. This helps to define an end-point for the updating procedure. The criteria may be based on whether a predetermined amount of data has been transmitted, and/or whether data has been transmitted for a predetermined time. Once the criteria has been reached, subsequent data transmitted to the base station is not used to update the current machine-learned model and is instead used in a subsequent machine-learned model updating procedure.

Throughout the present disclosure, machine-learned models can be or can otherwise include various machine-learned models such as artificial neural networks (e.g. deep neural networks) or other types of machine-learned models, including non-linear models and/or linear models. Neural networks can include feed-forward neural networks, recurrent neural networks (e.g. long short-term memory recurrent neural networks), convolutional neural networks or other forms of neural networks. Other examples of machine-learned models include Bayesian networks and Naïve Bayes networks. Other example machine-learned models/algorithms that may be used within the scope of the present disclosure include support vector machine techniques, Gaussian mixture models, hidden Markov models, decision trees, and genetic algorithms. Of course, other machine learning techniques as known to the skilled person may be used in the context of the present disclosure. The machine-learned model may be for performing activity classification, physiological classification, and/or biometric identification classification amongst other examples.

In examples of the present disclosure, the processors of the wearable article 20 and/or the base station 30 may comprise modules for performing signal processing and feature extraction of signals sensed by the wearable articles prior to employing the machine-learned model.

At least some of the example embodiments described herein may be constructed, partially or wholly, using dedicated special-purpose hardware. Terms such as ‘component’, ‘module’ or ‘unit’ used herein may include, but are not limited to, a hardware device, such as circuitry in the form of discrete or integrated components, a Field Programmable Gate Array (FPGA) or Application Specific Integrated Circuit (ASIC), which performs certain tasks or provides the associated functionality. In some embodiments, the described elements may be configured to reside on a tangible, persistent, addressable storage medium and may be configured to execute on one or more processors. These functional elements may in some embodiments include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. Although the example embodiments have been described with reference to the components, modules and units discussed herein, such functional elements may be combined into fewer elements or separated into additional elements. Various combinations of optional features have been described herein, and it will be appreciated that described features may be combined in any suitable combination. In particular, the features of any one example embodiment may be combined with features of any other embodiment, as appropriate, except where such combinations are mutually exclusive. Throughout this specification, the term “comprising” or “comprises” means including the component(s) specified but not to the exclusion of the presence of others.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. An electronics arrangement for a wearable article, the electronics arrangement comprising:
a removable electronics module configured to be releasably mechanically coupled to the wearable article, the electronics module comprising:
a plurality of processors, the plurality of processors comprising an application processor, and a hardware accelerator;
at least one memory storing instructions, the instructions, when executed by the plurality of processors, cause the plurality of processors to perform operations comprising:
(a) obtaining a current version of a machine-learned model;
(b) obtaining first data from at least one sensor of the wearable article; and
(c) employing the current version of the machine-learned model to generate an inference using the first data.

2. The electronics arrangement as claimed in claim 1, wherein the at least one sensor is provided in the wearable article separately to the electronics module.

3. The electronics arrangement as claimed in claim 1, wherein the operations further comprise (d) determining whether to update the current version of the machine-learned model by comparing a confidence level of the generated inference to a first predetermined threshold.

4. The electronics arrangement as claimed in claim 1, further comprising a communicator, and the operation (a) further comprises controlling the communicator to receive the current version of the machine-learned model from an external computer apparatus.

5. The electronics arrangement as claimed in any preceding claim 3, further comprising a communicator, wherein if the confidence level of the generated inference is greater than or equal to the first predetermined threshold, the operations further comprise (e) controlling the communicator to transmit the first data and the generated inference for the first data to a base station for the wearable article.

6. The electronics arrangement as claimed in claim 3, wherein if the confidence level of the generated inference is greater than or equal to the first predetermined threshold, the operations further comprise (e) updating the current version of the machine-learned model using the first data obtained from the wearable article.

7. The electronics arrangement as claimed in claim 6, wherein updating the current version of the machine-learned model comprises updating the machine-learned model using a backwards propagation of errors approach, a weight imprinting approach, and/or a transfer learning approach.

8. The electronics arrangement as claimed in claim 6, further comprising a communicator, wherein the operations further comprise (f) controlling the communicator to transmit updated machine-learned model data to an external computer apparatus.

9. The electronics arrangement as claimed in claim 1, wherein the first data comprises activity data sensed by the at least one sensor of the wearable article, and wherein the generated inference comprises an activity classification.

10. The electronics arrangement as claimed in claim 1, wherein the first data comprises physiological data sensed by the at least one sensor of the wearable article, and wherein the generated inference comprises a physiological classification.

11. The electronics arrangement as claimed in claim 1, wherein the first data comprises biometric identification data sensed by the at least one sensor of the wearable article, and wherein the generated inference comprises a biometric identification classification.

12. The electronics arrangement as claimed in claim 1, further comprising the at least one sensor, wherein the at least one sensor comprises at least one of an optical sensor, force sensor, electrical sensor, temperature sensor, and acoustic sensor.

13. The electronics arrangement as claimed in claim 12, wherein the optical sensor comprises a photoplethysmographic (PPG) sensor.

14. The electronics arrangement as claimed in claim 12, wherein the force sensor comprises at least one of an accelerometer, a magnetometer and a gyroscope.

15. The electronics arrangement as claimed in claim 12, wherein the electrical sensor comprises at least one of an electropotential sensor and an electroimpedance sensor, wherein the electropotential sensor comprises electrocardiogram (ECG), sensor and/or a electromyography (EMG) sensor, and wherein the electroimpedance comprises a skin conductance sensor.

16. (canceled)

17. (canceled)

18. The electronics arrangement as claimed in claim 1, wherein the hardware accelerator is arranged to employ at least a component of the machine-learned model.

19. (canceled)

20. A wearable article comprising the electronics arrangement as claimed in claim 1.

21. (canceled)

22. A base station for a wearable article, the base station comprising:
a plurality of processors, the plurality of processors comprising an application processor, and a hardware accelerator;
a mounting arrangement for forming a releasable mechanical coupling with at least a component of the wearable article; and
at least one memory storing instructions, the instructions, when executed by the plurality of processors, cause the plurality of processors to perform operations comprising:
(a) obtaining a current version of a machine-learned model;
(b) obtaining first data from at least one sensor of the wearable article; and
(c) employing the current version of the machine-learned model to generate an inference using the first data.

23. (canceled)

24. The base station as claimed in claim 22, further comprising a power source, and wherein the base station is arranged to transfer power from the power source to at least a component of the wearable article.

25. The base station as claimed in claim 22, wherein the base station further comprises a power transmitter arranged to wirelessly transfer power to a power receiver of at least a component of the wearable article.
